(19) United States
(12) Patent Application Publication
(10) Pub. No.: US 2026/0088197 A1
(43) Pub. Date: Mar. 26, 2026

(54) METHOD FOR PRODUCING A BI- OR MULTIPOLAR LEAD

(71) Applicants: Heraeus Medical Components LLC, Fridley, MN (US); Heraeus Medevio GmbH & Co. KG, Hanau (DE)

(72) Inventors: Leoni WILHELM, Hanau (DE); Markus JUNG, Hanau (DE); Richard W. KONISZCZUK, Fridley, MN (US); Andreas REISINGER, Hanau (DE)

(73) Assignees: Heraeus Medical Components LLC, Fridley, MN (US); Heraeus Medevio GmbH & Co. KG, Hanau (DE)

(21) Appl. No.: 19/337,279

(22) Filed: Sep. 23, 2025

Related U.S. Application Data
(60) Provisional application No. 63/698,317, filed on Sep. 24, 2024.

(51) Int. Cl. H01B 13/14; A61B 5/25; H01B 3/44; H01B 13/00

(52) U.S. Cl. CPC H01B 13/14; A61B 5/25; H01B 3/445; H01B 13/0036; A61B 2562/125; A61B 2562/222

(57) ABSTRACT

One aspect relates to a method for producing a multipolar lead for a medical device. A hollow-cylindrical inner lining is provided having a length. Electrically conductive channels are formed by an insulated conductor with a conductor and an insulating layer of a first thermoplastic having a first melting point. The electrically conductive channels are spirally wound around the inner lining to provide a cable. A second thermoplastic is coaxially extruded having a second melting point around the cable as outer insulation. The insulated cable is cut to provide an insulated lead cable. A plurality of contact openings is created in a distal end. The outer insulation and the insulating layer are removed to contact the conductive channel. Electrodes are provided and contacted with the conductive channels via the contact openings. The first melting point and the second melting point have a temperature difference of greater than or equal to 30°C.

### CROSS-REFERENCE TO RELATED APPLICATIONS

This application claims the benefit of and priority to U.S. Application No. 63/698,317, having a filing date of September 24, 2024, which is hereby incorporated by reference.

One aspect relates to a method for producing a bi- or multipolar lead for a medical device, comprising the method steps of:

a) providing a hollow-cylindrical inner lining having an inner lining length which corresponds to a multiple of a lead length of the lead for the medical device;

b) providing a plurality of electrically conductive channels, wherein each of the channels is formed by at least one insulated conductor comprising an electrical conductor and an insulating layer comprising a first thermoplastic having a first melting point T1;

c) spirally winding the electrically conductive channels around the inner lining to provide a cable having a cable length which corresponds to a multiple of the lead length;

d) coaxially extruding a second thermoplastic having a second melting point T2 around the cable to provide an insulated cable having the extruded second thermoplastic as an outer insulation;

e) cutting the insulated cable to a length that corresponds to the lead length, to provide an insulated lead cable;

f) providing a plurality of electrodes;

g) creating a plurality of contact openings in the vicinity of a distal end of the insulated lead cable, wherein, in order to create a contact opening, parts of the outer insulation and parts of the insulating layer of at least one of the electrically conductive channels are removed so that the at least one electrically conductive channel can be selectively contacted via each of the contact openings;

h) electrically contacting the plurality of electrodes with the plurality of electrically conductive channels via the plurality of contact openings, wherein each of the electrodes is selectively electrically connected to at least one of the electrically conductive channels via one of the contact openings; wherein the first melting point T1 and the second melting point T2 have a temperature difference DT of greater than or equal to 30°C.

The bi- or multipolar lead is suitable for a medical device and, in one embodiment, for a temporary measuring lead. However, the bi- or multipolar lead can also be used in other technical areas.

### BACKGROUND

Bi- or multipolar leads, and especially multipolar leads, for medical devices are usually very complex to produce since their production requires a large number of different components which must be assembled in a multitude of individual steps. The production of small-format leads, which are particularly in demand in the field of medical devices, is even more complex. The complex design of bi- or multipolar leads and especially their production usually lead to high prices for the final product.

The high prices of known bi- or multipolar leads are particularly problematic when the leads are to be used in medical devices for a relatively short period of time, e.g. in a temporary device for medical tests. In order to conduct temporary medical tests in an economically viable manner, cheaper unipolar leads are therefore chosen for the tests. However, the use of a unipolar lead may limit the success of the test, which may result in fewer patients receiving appropriate medical treatment in the form of a permanent medical device.

In addition, a bi- or multipolar lead designed and produced using a large number of individual components may demonstrate lower reliability because there are more possible failure modes of the individual parts. Thus, lower reliability may lead to a higher risk of failure of the lead, which in turn may have significant consequences, such as for a patient's health if the lead is used in a medical device.

In view of the above, it is desirable to provide a method for producing a bi- or multipolar lead which is simple, reliable, flexible and efficient to carry out.

Furthermore, it is desirable that the method allows the production of a mechanically stable and at the same time flexible bi- or multipolar lead. In particular, the resulting bi- or multipolar lead should be bendable without kinking or breaking its electrically conductive channels.

### OBJECTS

One object of the present invention is to overcome, at least in part, one or more of the disadvantages resulting from the prior art.

It is a further object of the invention to provide a method for producing a bi- or multipolar lead which is as simple, reliable, flexible and cost-effective as possible.

Furthermore, the resulting bi- or multipolar lead should be as mechanically robust as possible so that bending, in particular bending due to proper use in the tissue of a patient, is possible without mechanical damage to the electrically conductive channels of the bi- or multipolar lead.

### EMBODIMENTS

A contribution to the at least partial fulfillment of at least one of the aforementioned objects is made by the features of the independent claims. The dependent claims provide preferred embodiments that contribute to the at least partial fulfillment of at least one of the objects.

A first embodiment of the invention is a method for producing a bi- or multipolar lead for a medical device comprising the method steps of:

a) providing a hollow-cylindrical inner lining having an inner lining length which corresponds to a multiple of a lead length of the lead for the medical device;

b) providing a plurality of electrically conductive channels, wherein each of the channels is formed by at least one insulated conductor comprising an electrical conductor and an insulating layer comprising a first thermoplastic having a first melting point T1;

c) spirally winding the electrically conductive channels around the inner lining to provide a cable having a cable length which corresponds to a multiple of the lead length;

d) coaxially extruding a second thermoplastic having a second melting point T2 around the cable to provide an insulated cable having the extruded second thermoplastic as an outer insulation;

e) cutting the insulated cable to a length that corresponds to the lead length, to provide an insulated lead cable;

f) providing a plurality of electrodes, in particular at least two electrodes;

g) creating a plurality of contact openings in the vicinity of a distal end of the insulated lead cable, wherein, in order to create a contact opening, parts of the outer insulation and parts of the insulating layer of at least one of the electrically conductive channels are removed so that the at least one electrically conductive channel can be selectively contacted via each of the contact openings;

h) electrically contacting the plurality of electrodes with the plurality of electrically conductive channels via the plurality of contact openings, wherein each of the electrodes is selectively electrically connected to at least one of the electrically conductive channels via one of the contact openings; wherein the first melting point T1 and the second melting point T2 have a temperature difference DT of greater than or equal to 30°C.

In a preferred embodiment of the method, the first melting point T1 is higher than the second melting point T2. This embodiment is a second embodiment of the invention, which is preferably dependent upon the first embodiment of the invention.

In a preferred embodiment of the method, the first melting point T1 is in a range between 230°C and 290°C. This embodiment is a third embodiment of the invention, which preferably depends on the first or second embodiment of the invention.

In a preferred embodiment of the method, the second melting point T2 is in a range between 150°C and 260°C, preferably in a range between 160°C and 220°C. This embodiment is a fourth embodiment of the invention, which preferably depends on one of the previous embodiments of the invention.

In a preferred embodiment of the method, the first thermoplastic comprises a fluorinated polymer or, preferably, the first thermoplastic consists of a fluorinated polymer. This embodiment is a fifth embodiment of the invention, which preferably depends upon one of the previous embodiments of the invention.

In a preferred embodiment of the method, the fluorinated polymer is an ethylene-tetrafluoroethylene copolymer (ETFE), a perfluoroalkoxy polymer (PFA), a polyvinylidene fluoride (PVDF), a tetrafluoroethylene-hexafluoropropylene copolymer (FEP) or a mixture of an ethylene-tetrafluoroethylene copolymer, a perfluoroalkoxy polymer, a polyvinylidene fluoride or a tetrafluoroethylene-hexafluoropropylene copolymer. This embodiment is a sixth embodiment of the invention, which is preferably dependent upon the fifth embodiment of the invention.

In a preferred embodiment of the method, the second thermoplastic is a polyurethane (PU). This embodiment is a seventh embodiment of the invention, which preferably depends upon one of the preceding embodiments of the invention.

In a preferred embodiment, the polyurethane is an aromatic polyurethane based on polyether or polyester, preferably based on polyether. This embodiment is an eighth embodiment of the invention, which is preferably dependent on the seventh embodiment of the invention.

In a preferred embodiment of the method, the inner lining comprises a polytetrafluoroethylene (PTFE) or preferably, the inner lining consists of a polytetrafluoroethylene. This embodiment is a ninth embodiment of the invention, which preferably depends upon one of the preceding embodiments of the invention.

In a preferred embodiment of the method, the extrusion in method step d) is carried out in a temperature range between 170°C and 220°C, preferably between 180°C and 215°C. This embodiment is a tenth embodiment of the invention, which preferably depends upon one of the preceding embodiments of the invention.

In a preferred embodiment of the method, the provision in method step b) comprises coaxially extruding the first thermoplastic around the electrical conductor to form the insulating layer. This embodiment is an eleventh embodiment of the invention, which preferably depends upon one of the preceding embodiments of the invention.

In a preferred embodiment of the method, the coaxial extrusion of the first thermoplastic around the electrical conductor is carried out in a temperature range between 250°C and 300°C. This embodiment is a twelfth embodiment of the invention, which is preferably dependent upon the eleventh embodiment of the invention.

### GENERAL

With respect to the embodiments described herein, the elements of which "have," or "comprise," a particular feature (for example, a material), in principle, a further embodiment is always contemplated in which the relevant element consists solely of the feature, i.e., does not comprise any other constituents. The word "comprise" or "comprising" is used herein synonymously with the word "have" or "having."

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, it is contemplated in principle that all features described herein in connection with a method are also applicable to the products and devices described herein, and vice versa.

All such considered combinations are not explicitly listed in all instances, simply in order to keep the description brief. Technical solutions known to be equivalent to the features described herein are also intended in principle to be encompassed by the scope of the invention.

In the present description, specifications of ranges also contain the values specified as limits. A specification of the type "in the range from X to Y" with respect to a quantity A consequently means that A can take the values X, Y and values between X and Y. Ranges which are limited on one side, of the type "up to Y" for a size A, accordingly mean a value Y and less than Y.

Some of the features described are associated with the term "substantially." The term "substantially" is to be understood in such a way that, under real conditions and manufacturing techniques, a mathematically exact interpretation of terms such as "superimposition," "perpendicular," "diameter" or "parallelism" can never be given exactly, but only within certain manufacturing error tolerances. For example, "substantially perpendicular axes" enclose an angle of 85 degrees to 95 degrees relative to one another, and "substantially equal volumes" comprise a variation of up to 5% by volume. For example, a "device consisting substantially of plastics" comprises a plastics content of ≥95 to ≤100% by weight. For example, a "substantially complete filling of a volume B" comprises a filling of ≥95 to ≤100% by volume of the total volume of B.

When an indefinite or definite article is used when referring to a singular noun, such as "a," "an" or "the," it includes a plural of that noun unless explicitly stated otherwise. When the term “comprising” is used in the present description and claims, other elements are not thereby excluded.

For the purposes of the present invention, the terms “consisting substantially of” and “consisting of” are considered embodiments of the term “comprising”. When a group is defined below as comprising at least a certain number of embodiments, this is also to be understood as disclosing a group which, in one embodiment, consists substantially only of these embodiments or, in one embodiment, consists only of these embodiments.

Terms such as “obtainable” or “definable” and “obtained” or “defined” are used interchangeably. This means, for example, that the term "obtained" does not imply that an embodiment must be obtained, for example, by the sequence of steps following the term "obtained," unless the context clearly dictates otherwise, although such a limited understanding is always included in the terms "obtained" or "defined" as an embodiment. Whenever the terms “including” or “with” are used, these terms are synonymous with “comprising” as defined above.

### DETAILED DESCRIPTION

A first object of the invention relates to a method for producing a bi- or multipolar lead for a medical device, comprising the method steps of:

a) providing a hollow-cylindrical inner lining having an inner lining length which corresponds to a multiple of a lead length of the lead for the medical device;

b) providing a plurality of electrically conductive channels, wherein each of the channels is formed by at least one insulated conductor comprising an electrical conductor and an insulating layer comprising a first thermoplastic having a first melting point T1;

c) spirally winding the electrically conductive channels around the inner lining to provide a cable having a cable length which corresponds to a multiple of the lead length;

d) coaxially extruding a second thermoplastic having a second melting point T2 around the cable to provide an insulated cable having the extruded second thermoplastic as an outer insulation;

e) cutting the insulated cable to a length that corresponds to the lead length, to provide an insulated lead cable;

f) providing a plurality of electrodes;

g) creating a plurality of contact openings in the vicinity of a distal end of the insulated lead cable, wherein, in order to create a contact opening, parts of the outer insulation and parts of the insulating layer of at least one of the electrically conductive channels are removed so that the at least one electrically conductive channel can be selectively contacted via each of the contact openings;

h) electrically contacting the plurality of electrodes with the plurality of electrically conductive channels via the plurality of contact openings, wherein each of the electrodes is selectively electrically connected to at least one of the electrically conductive channels via one of the contact openings; wherein the first melting point T1 and the second melting point T2 have a temperature difference DT of greater than or equal to 30°C.

The method is used to produce a bi- or multipolar lead for a medical device in a simple, efficient manner, wherein due to the temperature difference DT between the first melting point T1 and the second melting point T2 of 30°C or more, there is no integral bond between the insulating layer of the electrically conductive channels and the outer insulation. This gives the turns of the conductive channels, which are created by the spiral winding in method step c) and which are arranged between the inner lining and the outer insulation, a certain freedom of movement which reduces the risk of kinking or even breaking off during proper bending of the bi- or multipolar lead. The windings of the electrically conductive channels can, for example, be stretched or compressed during bending of the bipolar or multipolar lead, which can lead to a mechanical relief of the electrically conductive channels. If the insulating layers were connected in an integrally bonded manner to the outer insulation, this freedom of movement would be significantly restricted, and the risk of bending or breaking off would be significantly increased.

Furthermore, due to high flexibility, the method also allows for a plurality of different leads, in particular leads of different lengths and/or leads provided with a different number of electrodes, which makes these types of bi- or multipolar leads easily and efficiently accessible. The method also enables electrical contact of the electrodes, particularly in the form of ring electrodes, at any point of the insulated lead cable. Due to the efficient implementation of the method, leads can also be produced for temporary applications, such as temporary medical measuring arrangements, which would not be attractive from an economical perspective using other methods.

A “bipolar” lead in the sense of the invention is understood to mean a lead comprising two electrodes which are each electrically connected to one or more electrically conductive channels near the distal end of the lead cable. A “multipolar” lead in the sense of the invention is understood to mean a lead comprising at least three electrically conductive channels and three electrodes which are each electrically connected to one of the conductive channels near the distal end of the lead cable. In general, the polarity of the lead is determined by the number of conductive channels in the lead. Since each of the conductive channels is selectively connected to an electrode near the distal end of the lead cable, the number of electrodes also determines the minimum number of electrically conductive channels near the distal end of the lead cable. Thus, a multipolar lead comprising four electrodes comprises a minimum of four electrically conductive channels.

“Near the distal end” of the lead cable means that a position lies within the last 30%, preferably the last 20%, most preferably the last 10% of the lead cable length, and “near the proximal end” means that a position lies within the first 30%, preferably the first 20%, most preferably the first 10% of the lead cable length.

An “electrically conductive channel” in the sense of the invention is a means for selectively electrically connecting an electrical part (e.g. a ring electrode) near the proximal end of the lead to an electrode, preferably a ring electrode, near the distal end of the lead.

In the context of the invention, the term “thermoplastic” refers to a class of polymers that are characterized by a particular thermal behavior. Thermoplastics are materials that soften or melt when heated to a specific temperature, their melting point, and solidify again when cooled. This property enables repeated shaping through thermal processes such as extrusion, injection molding or thermoforming.

In the context of the invention, the term “melting point” is used to describe the phenomenon in which the polymer under consideration, in particular the thermoplastic under consideration, changes from a solid to a liquid state. It should be noted that this melting point should not necessarily be understood as a discrete temperature, but in certain cases it can also be a melting range. The melting range refers to a temperature interval in which the polymer, especially the thermoplastic, begins to melt and progressively loses its solid structure until it has completely changed into the liquid state. This understanding takes into account the fact that many polymers, especially thermoplastics, especially those with a semi-crystalline, heterogeneous structure, do not exhibit a uniform melting point, but rather a range in which the melting processes take place successively.

In method step a), a hollow-cylindrical, i.e. in the broadest sense hose- or tube-shaped, inner lining is provided. The inner lining defines an inner lumen, i.e., a free space, preferably in the middle of the lead, which extends axially through the lead to be manufactured. The inner lumen represents a cavity in the lead to be manufactured into which further devices such as guide wires can be introduced and/or through which further devices can be introduced through the lead into a patient's body. The inner lining serves as a support structure for the lead and can give it improved stability.

The inner lining can, for example, comprise or consist of a hollow cylinder, for example made of a polymer, a fabric, for example made of a metal, a braid, for example made of a metal, and/or a spiral or helix, both made of a metal, for example. For example, the inner lining consists of a tube made of a polymer, the wall of which is mechanically reinforced with a spiral made of a metal.

The inner lining has an inner lining length which is a multiple of, for example five to two hundred times, the length of the lead to be manufactured. Such a long inner lining is therefore suitable for the production of a large number of leads. This simplifies the production process and also allows leads of different lengths to be produced from the same starting material, which makes the production process flexible.

The inner lining preferably comprises an outer diameter in a range of 50 µm to 500 µm, in one embodiment in a range of 200 µm to 400 µm, and a wall thickness in a range of 10 µm to 100 µm and in one embodiment of 20 µm to 80 µm.

In method step b), a plurality of electrically conductive channels are provided, for example from two to thirty, preferably from four to twenty, most preferably from six to fourteen. Each of the channels comprises at least one insulated conductor comprising an electrical conductor and an insulating layer. The insulating layer serves to electrically isolate the electrically conductive channel from another electrically conductive channel.

The electrical conductor of the at least one insulated conductor may be a single conductor or a plurality of individual conductors. Preferably, the conductor is a metal wire or a plurality of metal wires, wherein the plurality of metal wires is preferably wound, stranded, braided or rolled into a bundle of metal wires. The metal wire or the metal wire bundle may have a diameter in a range of 5 to 250 µm, preferably in a range of 10 to 120 µm. For example, the conductor (e.g. the metal wire or the metal wire bundle) may have a diameter of about 85 µm.

The electrical conductor preferably consists of a metal selected from the group that consists of platinum, iridium, tantalum, palladium, titanium, iron, gold, molybdenum, niobium, tungsten, nickel, chromium, cobalt, stainless steel, nitinol, alloys of any of these metals and composite materials, for example in the form of a sheathed wire, of any of these metals. Stainless steel such as stainless steel AISI 316L, stainless steel AISI 301 or stainless steel AISI 304 is suitable as an electrical conductor. Platinum and platinum alloys such as Pt/Ir 10 or Pt/Ir 20 are suitable as electrical conductors. Nickel-cobalt alloys such as MP35N are suitable as electrical conductors.

The electrical conductor can also be provided with a coating (e.g. platinum) or plated to increase corrosion resistance. The electrical conductor can be, for example, a Pt-coated MP35N conductor or a Pt-coated tungsten-based conductor.

The insulating layer of the electrically conductive channels comprises a first thermoplastic with a melting point T1. Preferably, the insulating layer of the electrically conductive channels consists of a first thermoplastic with a melting point T1.

In method step c), the electrically conductive channels are spirally wound around the inner lining to provide a cable. Due to the winding, the inner lining is preferably coaxially surrounded by turns of the electrically conductive channels. The cable provided in this way has a cable length which corresponds to a multiple of the lead length; in particular, the length of the cable substantially corresponds to the inner lining length of the inner lining provided in method step a). Preferably, the electrically conductive channels are wound spirally around the inner lining, lying next to one another. More preferably, the electrically conductive channels in the wound state extend substantially from a proximal inner lining end to a distal inner lining length of the inner lining.

The advantage of spiral winding is that it can achieve improved stability and flexibility of the lead. Furthermore, the arrangement of the electrically conductive channels in turns allows a certain compression or stretching of these turns when bending the inner lining and later also the final produced cable.

In method step d), a second thermoplastic with a second melting point is coaxially extruded around the cable. As a result, the cable is encased in the second thermoplastic, and the second thermoplastic acts, at the latest after it has cooled down, as an external insulation, in particular external electrical insulation. For the sake of clarity, it should be noted that the cable obtained in method step c) is already ‘insulated’ by the electrically conductive channels provided with the insulating layer. The term “insulated” in relation to the wording “insulated cables” refers to the presence of external insulation, which in particular is electrically insulating.

In method step e), the insulated cable is cut to a length which substantially corresponds to the lead length, while providing an insulated lead cable. The cutting also allows a plurality of insulated lead cables to be provided from a single insulated cable. The desired length of the lead is determined by the cutting. The insulated cable can be cut into pieces of equal length so that the lead length of each of the final leads is the same, or the insulated cable can be cut into different lead lengths so that leads of different lengths can be produced from the insulated cable. The cutting thus increases the flexibility of the method which can also be carried out cost-effectively.

The cutting can be carried out in different ways. For example, the cutting can be done using pliers, scissors or using a wire cutting machine. Furthermore, the cutting can be carried out using a laser, for example.

In method step f), a plurality of electrodes are provided for the lead. The exact number of electrodes depends on the use of the lead. Preferably, a maximum of as many electrodes as electrically conductive channels are provided so that each of the electrodes can be electrically connected to an electrically conductive channel during the method. More preferably, the number of electrodes is half the number of provided electrically conductive channels so that over the course of the method, each of the electrodes can be electrically connected to two electrically conductive channels. In this way, each electrode comprises a second electrically conductive channel as a backup system in case the first electrically conductive channel fails. Preferably, the electrodes are ring electrodes.

The electrodes can comprise a plurality of different materials or consist of different materials. Preferably, the electrodes comprise a metal that is selected from the group consisting of platinum, iridium, tantalum, palladium, titanium, iron, gold, molybdenum, niobium, tungsten, nickel, chromium, cobalt, steel, nitinol, alloys of any of these metals, and composites of any of these metals. Stainless steel is suitable as an electrode, for example stainless steel AISI 316L, stainless steel AISI 301 or stainless steel AISI 304. Platinum and platinum alloys such as Pt/Ir 10 or Pt/Ir 20 or nickel-cobalt alloys such as MP35N are also suitable as electrodes.

The choice of metal for the electrodes (and for the electrical conductors) may depend on the use of the lead according to the invention. For example, if the lead according to the invention is to be used in a permanent medical device, the electrodes may comprise, or in one embodiment consist of, platinum or a platinum-iridium alloy. If the lead according to the invention is to be used in a temporary medical device, the electrodes may comprise, and in one embodiment consist of, stainless steel. However, it should be understood that the application of the lead is not limited by the use of a particular metal.

The electrodes may also have a coating. Suitable coatings are metal nitrides such as TiN, metal oxides such as IrO₂ or conductive polymers. The surface of the electrodes can also be surface-structured, e.g., laser-structured.

Each of the electrodes, preferably ring electrodes, can have an outer diameter in the range of 200 to 5000 µm, in one embodiment in the range of 300 to 3000 µm and in one embodiment in the range of 500 to 1500 µm. Each of the electrodes may have a wall thickness in the range of 10 to 200 µm, in one embodiment 10 to 100 µm, and in one embodiment 30 to 70 µm. Furthermore, each of the electrodes may have a length in the range of 200 to 5000 µm, in one embodiment 300 to 3000 µm, and in one embodiment in the range of 500 to 1500 µm. According to one embodiment, each of the electrodes has an outer diameter in the range of 300 to 3000 µm and in one embodiment in the range of 500 to 1500 µm, a wall thickness in the range of 10 to 100 µm and in one embodiment of 30 to 70 µm, and a length in the range of 300 to 3000 µm and in one embodiment in the range of 500 to 1500 µm.

In method step g), a plurality of contact openings, preferably in the same number as the provided electrodes, are created in the vicinity of the distal end of the insulated lead cable. For this purpose, a part of the outer insulation, i.e., a part of the second thermoplastic, as well as a part of the insulating layer, i.e., a part of the first thermoplastic, of at least one of the electrically conductive channels running there is removed at the corresponding locations, so that at least one electrical conductor of an electrically conductive channel is accessible from the outside and can be electrically contacted at each of the contact openings. Preferably, only a radially outer part of the insulating layer of the electrical conductor is removed. At each of the contact openings, one of the provided electrodes can thus be electrically connected to at least one of the electrically conductive channels over the further course of the method. In one embodiment, parts of the insulating layer of two electrically conductive channels are removed at the contact openings so that one of the electrodes can be selectively electrically connected to two electrically conductive channels via these contact openings.

In the axial extension, the contact openings preferably comprise a length which substantially corresponds to the axial extension of the electrodes.

The contact opening can be created in different ways. For example, the opening can be created by cutting, punching or, preferably, by laser ablation.

In method step h), the plurality of electrodes are electrically contacted with the plurality of electrically conductive channels via the plurality of contact openings. Each of the electrodes is selectively, i.e., specifically, electrically connected to at least one of the electrically conductive channels via one of the contact openings.

The electrical contacting can be carried out in different ways. For example, a direct electrical contact can be created between the electrode and the electrically conductive channel by bringing both parts into direct electrical contact. Furthermore, an indirect electrical contact can be created by electrically connecting the electrode and the electrically conductive channel via an electrically conductive bridge element, i.e., a separate, electrically conductive component, such as a wire or a wire portion, comprising an electrically conductive material such as a metal or an electrically conductive polymer. The bridge element serves in particular to bridge the distance between the electrically conductive channel(s), in particular their electrical conductors, and the electrode to be contacted, which is caused in particular by the radial thickness of the outer insulation.

For example, the electrodes are ring electrodes which are pressed onto the electrical conductor of the electrically conductive channel by external force and are thus pressed together radially in order to establish an electrical contact, directly or indirectly.

Furthermore, in order to securely connect the electrode to the electrically conductive channel, a fastening such as a weld, preferably by means of laser action, can be formed between the electrode and the electrically conductive channel.

The first melting point T1 of the first thermoplastic and the second melting point of the second thermoplastic T2 have a temperature difference DT of at least 30°C, preferably of at least 40°C, more preferably of at least 50°C, more preferably of at least 60°C, most preferably of at least 70°C, wherein it is preferred that the first melting point T1 is higher than the second melting point T2. The first thermoplastic therefore melts preferably at a higher temperature than the second thermoplastic.

This allows the second thermoplastic to be extruded, preferably directly, onto the first thermoplastic without the two thermoplastics forming an integral bond or a mechanical connection in any other way.

For extrusion, the second thermoplastic should preferably be heated at least to its melting temperature T2 or at least to a temperature near the melting point T2. Furthermore, due to the spatial proximity of the two thermoplastics during extrusion of the second thermoplastic, preferably even via their direct contact during extrusion, heat is transferred from the second thermoplastic heated for extrusion to the first thermoplastic. However, the extrusion temperature is preferably set such that the heat transferred to the first thermoplastic is not sufficient to heat the first thermoplastic to the first melting temperature T1. Preferably, the extrusion temperature, i.e., the temperature to which the second thermoplastic is heated during extrusion, is below the first melting temperature T1, more preferably between the melting temperature T1 and the melting temperature T2.

The electrically conductive channels are therefore not connected to the outer insulation in such a way that a movement of the outer insulation is directly and immediately transmitted to the electrically conductive channels. This allows bending of the outer insulation, which can be compensated for at least to some extent by the electrically conductive channels by compressing and/or stretching the turns of the electrically conductive channels around the inner lining. This reduces the risk of kinking or even breaking the electrically conductive channels when using the bipolar or multipolar lead properly, especially in a patient's tissue.

A preferred embodiment of the method is characterized in that the first melting point T1 is in a range between 230°C and 290°C, preferably between 240°C and 285°C, more preferably between 245°C and 280°C. This temperature range allows a wide selection of second thermoplastics to be used, while the distance according to the invention between the melting temperatures T1 and T2 is maintained.

A preferred embodiment of the method is characterized in that the second melting point T2 is in a range between 150°C and 260°C, preferably between 160°C and 220°C.

This temperature range allows a wide selection of first thermoplastics to be used, while the distance according to the invention between the melting temperatures T1 and T2 is maintained.

The first thermoplastic can be selected from a variety of different thermoplastics or combinations of thermoplastics.

A preferred embodiment of the method is characterized in that the first thermoplastic comprises a fluorinated polymer, preferably consists of a fluorinated polymer. Fluorinated polymers are preferred because they usually have improved sliding properties, or in other words, a low coefficient of friction, compared to non-fluorinated polymers. This also reduces the risk of kinking or breaking the electrically conductive channels when properly bending the bipolar or multipolar lead.

A preferred embodiment of the method is characterized in that the fluorinated polymer is an ethylene-tetrafluoroethylene copolymer (ETFE), a perfluoroalkoxy polymer (PFA), a polyvinylidene fluoride (PVDF) and/or a tetrafluoroethylene-hexafluoropropylene copolymer (FEP).

The second thermoplastic can be selected from a variety of different thermoplastics or combinations of thermoplastics.

A preferred embodiment of the method is characterized in that the second thermoplastic is a polyurethane. Polyurethanes can usually be processed, especially extruded, at comparatively low temperatures.

A preferred embodiment of the method is characterized in that the polyurethane is a polyester-based or polyether-based polyurethane.

The inner lining may comprise or consist of different materials or combinations of materials. Preferably, the inner lining consists of a polymer.

A preferred embodiment of the method is characterized in that the inner lining comprises a fluorinated polymer. Preferably, the inner lining consists of a fluorinated polymer. Preferably, the fluorinated polymer of the inner lining is a polytetrafluoroethylene (PTFE). PTFE is preferred because it has a low coefficient of friction so that the turns of the electrically conductive channels are not restricted in their relative movement by the inner lining. Furthermore, an inner lining made of a fluorinated polymer, in particular ETFE, ensures that no integral bond is formed between the inner lining and an outer insulation made of an extruded polyurethane, which is preferred as the second thermoplastic. This additionally ensures the relative freedom of movement of the windings of the electrically conductive channels between the inner lining and the outer insulation.

The extrusion in method step d) can take place at different temperatures. Preferably, the extrusion takes place at an extrusion temperature which is lower than the first melting point T1. Further preferably, the extrusion takes place at an extrusion temperature which is higher than the second melting point T2 but lower than the first melting point T1, i.e., between the first melting point T1 and the second melting point T2. This effectively prevents an integral bond between the insulating layer of the electrically conductive channels and the outer insulation.

A preferred embodiment of the method is characterized in that the extrusion in method step d) is carried out in a temperature range between 170°C and 220°C.

The provision of the electrically conductive channels in method step b) can be done in different ways.

A preferred embodiment of the method is characterized in that the provision in method step b) comprises a coaxial extrusion of the first thermoplastic around the electrical conductor to form the insulating layer. It goes without saying that this coaxial extrusion of the first thermoplastic may have already taken place before the start of the method according to the invention. Extruding the first thermoplastic is a simple and cost-effective way to produce the plurality of electrically conductive channels.

The method conditions for coaxial extrusion of the first thermoplastic must be selected depending on the utilized first thermoplastic.

A preferred embodiment of the method is characterized in that the coaxial extrusion of the first thermoplastic around the electrical conductor, preferably around all electrical conductors, is carried out in a temperature range between 250°C and 300°C.

### Examples

The invention will now be explained in greater detail using an exemplary method. The invention is not limited by the example.

To produce an exemplary lead according to the invention, a 500 m long inner lining in the form of a polymer tube made of PTFE with an inner diameter of 400 µm and an outer diameter of 450 µm was provided.

Furthermore, 12 electrically conductive channels with a length of 1000 m each were provided. Each of the 12 electrically conductive channels consisted of a 1x7 microcable with microcable wires made of MP35N with a silver core as the electrical conductor. Each of the electrical conductors was coated with ETFE as a first thermoplastic as the insulating layer. The first melting point T1 of the first thermoplastic used was 263°C. The single electrically conductive channel had an outer diameter of 150 µm, with the thickness of the insulating layer being 30 µm.

The 12 electrically conductive channels were wound spirally around the inner lining, lying next to one another, using a stranding machine, substantially wrapping around the entire length of the inner lining.

The cable obtained by spirally winding the electrically conductive channels around the inner lining was provided with a jacket as an outer insulation by coaxially extruding a polyurethane (Pellethane® 236355DE, available from Lubrizol Corporation, USA) as a second thermoplastic with a second melting point T2 of 210°C. Extrusion was carried out at an extrusion temperature of 212°C. The thickness of the outer insulation was 200 µm.

The insulated cable obtained by extruding the second thermoplastic was cut into individual insulated lead cables each 90 cm long using a wire cutting machine.

Subsequently, 6 ring electrodes made of Pt/Ir 10 (platinum-iridium alloy with 10 weight percent iridium based on the total mass of the alloy) with a wall thickness of 200 µm were provided.

By means of laser ablation, six disjoint contact openings were created near the distal end of the insulated lead cable so that two electrically conductive channels per contact opening could be selectively electrically contacted with one of the electrodes each. The axial extension of the contact opening was adapted to the wall thickness of the electrodes.

To electrically contact the electrodes, they were pushed onto the cable at the corresponding positions of the contact openings, pressed together radially and fixed to the corresponding electrical conductors in an electrically conductive manner by means of laser welding.

### FIGURES

The invention is further illustrated by way of example below by means of figures. The invention is not limited to the figures.

In the figures,

FIG. 1 shows an exemplary flow chart of a method for producing a bi- or multipolar lead for a medical device, and

FIGS. 2a-f show exemplary intermediate products of the method for producing a multipolar lead in a schematic cross section.

### DESCRIPTION OF THE FIGURES

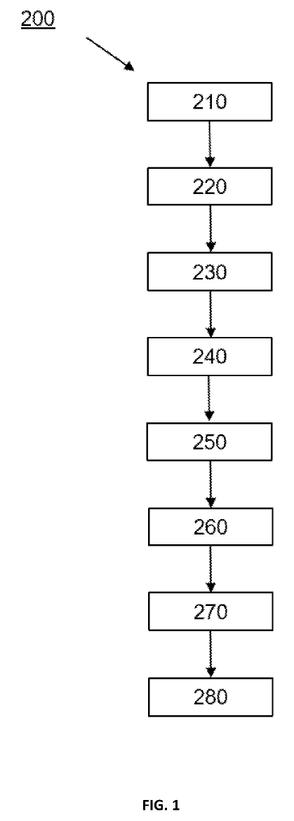

FIG. 1 shows a flow chart of an exemplary method for producing a bi- or multipolar lead for a medical device comprising the method steps 210 to 280.

In a method step 210, a hollow cylindrical inner lining is provided, with an inner lining length which corresponds to a multiple of a final lead length of the lead to be produced for the medical device.

In a method step 220, a plurality of electrically conductive channels are provided. Each of the channels comprises at least one insulated conductor comprising an electrical conductor and an insulating layer arranged around the electrical conductor. The insulating layer comprises a first thermoplastic having a first melting point T1, preferably the insulating layer consists of a first thermoplastic having a first melting point T1.

In a method step 230, the plurality of electrically conductive channels provided in method step 220 are spirally wound around the inner lining provided in method step 210. Method step 230 thus provides a cable, wherein a cable length of the cable corresponds to a multiple of the lead length, in particular wherein the cable length corresponds to the inner lining length.

In a method step 240, a second thermoplastic having a second melting point is coaxially extruded around the cable obtained in method step 240 to form an insulated cable having the extruded second thermoplastic as an outer insulation. Preferably, the second thermoplastic is extruded over substantially the entire length of the cable. There is a temperature difference between the first melting point T1 and the second melting point T2 DT of at least 30°C. Preferably, the first melting point T1 is higher than the second melting point T2. The extrusion preferably takes place at an extrusion temperature which is lower than the first melting point 1, more preferably at an extrusion temperature which is between the first melting point T1 and the second melting point T2.

In a method step 250, the insulated cable obtained in method step 230 is cut to a length that corresponds to the lead length. Due to the cutting, a plurality of insulated lead cables are preferably obtained, wherein the insulated lead cables differ from the insulated cable substantially only in their length. The insulated lead cables thus obtained preferably have the same length.

In a method step 260, a plurality of electrodes are provided. Preferably, the electrodes are ring electrodes. The number of electrodes depends on the application of the lead to be produced. Preferably, the number of electrodes corresponds to a maximum of the number of electrically conductive channels used in the method.

In a method step 270, a plurality of contact openings are created in a vicinity of a distal end of the insulated lead cable. The contact openings serve to electrically contact the electrodes with the electrically conductive channels. For this purpose, a part of the outer insulation and a part of the insulating layer of at least one electrically conductive channel are removed at each of the contact openings, so that an electrode can be selectively electrically contacted with the corresponding electrical conductor of the electrically conductive channel via the contact opening. Exactly one or more than one, for example two or three, electrically conductive channels can be electrically connected to an electrode via a contact opening. Preferably, the number of contact openings corresponds to the number of electrodes, so that one electrode can be electrically contacted via each contact opening.

For example, the contact openings can be created by laser ablation.

In a method step 280, the plurality of electrodes are electrically contacted with the plurality of electrically conductive channels via the contact openings. Each of the electrodes is selectively electrically contacted via one of the contact openings with one or more electrically conductive channels which are accessible via the respective contact opening.

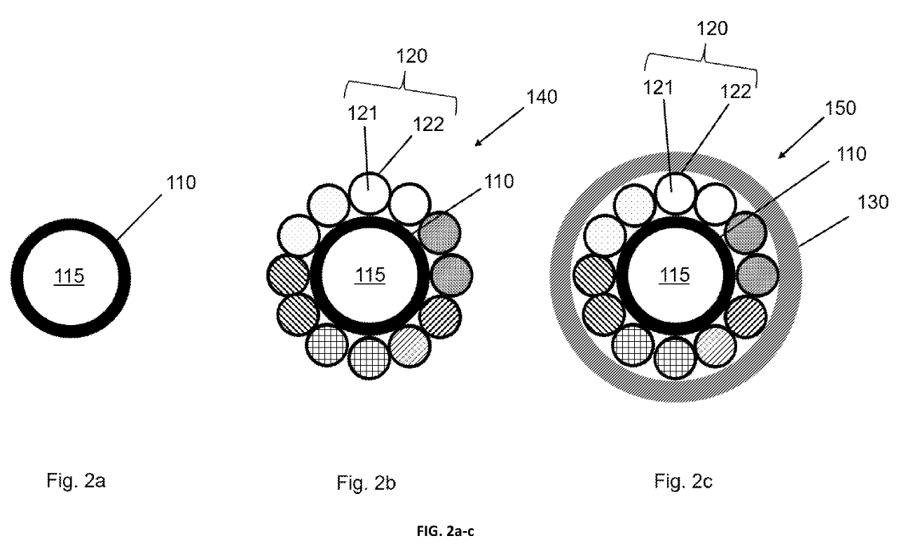

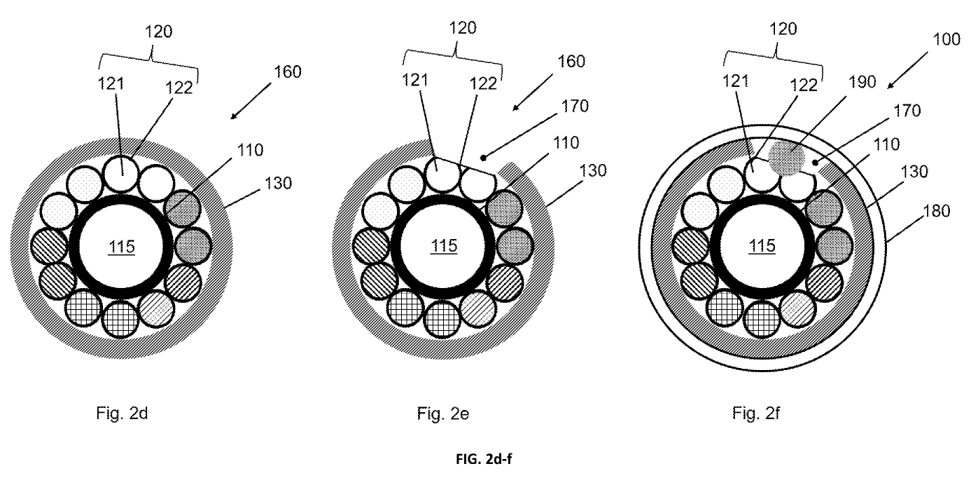

FIGS. 2a-f show various exemplary intermediate products of the method 200 shown in FIG. 1 for producing a bi- or multipolar lead in a schematic cross section.

FIG. 2a shows a hollow-cylindrical inner lining 110 consisting of a polytetrafluoroethylene. The inner lining 110 encloses an inner lumen 115 and has an inner lining length which corresponds to a multiple of a lead length of the final produced lead (not visible in the cross section).

FIG. 2b shows a cable 140 comprising the inner lining 110 from FIG. 2a and a total of twelve electrically conductive channels 120 (provided with reference signs only as an example), wherein the twelve electrically conductive channels 120, which were previously provided, were wound spirally around the inner lining 11. In the embodiment shown, each of the electrically conductive channels 120 comprises exactly one insulated conductor which comprises an electrical conductor 121 with an insulating layer 122 surrounding the electrical conductor 121 and made of a first thermoplastic with a first melting point T1. The electrically conductive channels 120 are each shown in adjacent groups of two with different hatching, which is only intended to provide a better overview of the intermediate products shown. Otherwise, the electrically conductive channels 120 do not differ from each other. The electrically conductive channels 120 are wound spirally (not visible in the cross section) and adjacently to one another along the entire inner lining length 110.

FIG. 2c shows an insulated cable 150 obtained by coaxially extruding a second thermoplastic having a second melting point T2 around the cable 140 of FIG. 2b. The second thermoplastic forms an outer insulation 130 of the insulated cable 150. The first melting point T1 and the second melting point T2 are selected by selecting the first thermoplastic and the second thermoplastic in such a way that no connection, in particular no integral bond, occurs between the two thermoplastics during coaxial extrusion. In this way, the wound electrically conductive channels 120, in particular in the axial extension, have a certain freedom of movement so that they have a reduced risk of breaking or kinking when the final lead is bent correctly. In particular, it is possible to compress or stretch the turns. In the embodiment shown, the first melting point T1 is at least 40°C higher than the second melting point T2. Furthermore, the coaxial extrusion was carried out at an extrusion temperature which is lower than the first melting point T1 and in particular between the first melting point T1 and the second melting point T2.

FIG. 2d shows an insulated lead cable 160 obtained by cutting the insulated cable 150 from FIG. 2c. The cutting was done to the desired lead length (not visible in the cross section) which the final produced lead should have. The cutting was done using a wire cutting machine.

FIG. 2e shows the insulated lead cable 160 from FIG. 2d, wherein, in contrast to FIG. 2d, the insulated lead cable 160 comprises a contact opening 170 in the vicinity of a distal end (not visible in the cross section). The contact opening 170 was created by removing parts of the outer insulation 130 and parts of the insulating layer 122 of two adjacent electrically conductive channels 120. By removing these electrically insulating components, selective electrical contacting of the electrical conductors 121 of the corresponding electrically conductive channels 120 from outside the insulated lead cable 160 is possible. In the shown embodiment shown, a contact opening 170 comprises exposing two adjacent electrical conductors 121 so that they can be electrically contacted with a single electrode. This increases the reliability of the final conductor in the event of a failure, for example due to severing, of one of the electrical conductors 121, in particular due to kinking. In this case, a current flow would still be possible via the second electrically conductive channel 120.

FIG. 2f shows the final lead 100 produced using the method 200. Starting from the intermediate product of FIG. 2e, the two adjacent electrically conductive channels 120, in particular their electrical conductor 121, were electrically contacted with an electrode 180 in the form of a ring electrode. In the embodiment shown, this was not done by direct but rather by indirect electrical contact via a bridge element 190 in the form of an electrically conductive wire portion. The bridge element 190 serves in particular to bridge the distance between the electrical conductors 121 and the electrode 180, which is mainly caused by the radial thickness of the outer insulation 135. The lead 100 comprises a total of six electrodes 180, each of which selectively electrically contacts two adjacent electrically conductive channels 120 via a total of six contact openings 170, wherein only one electrode 180 including the contact point is visible in the cross section shown. Each of the electrically conductive channels 120 is electrically connected only to a single electrode 180 indirectly via a bridge element 190.

The features disclosed in the claims, the description and the drawings may be essential for various embodiments of the claimed invention both individually and in any combination with one another.

### REFERENCE SIGNS

100 lead

110 inner lining

115 inner lumen

120 electrically conductive channel

121 electrical conductor

122 insulating layer

130 outer insulation

140 cable

150 insulated cable

160 insulated lead cable

170 contact opening

180 electrode

190 bridge element

200 method

210 provision of inner lining

220 provision of electrically conductive channels

230 winding

240 coaxial extrusion

250 cutting

260 provision of electrodes

270 creation of contact openings

280 contacting

1. A method for producing a bi- or multipolar lead for a medical device, comprising:
a) providing a hollow-cylindrical inner lining having an inner lining length which corresponds to a multiple of a lead length of the lead for the medical device;
b) providing a plurality of electrically conductive channels, wherein each of the channels is formed by at least one insulated conductor comprising an electrical conductor and an insulating layer comprising a first thermoplastic having a first melting point T1;
c) spirally winding the electrically conductive channels around the inner lining to provide a cable having a cable length which corresponds to a multiple of the lead length;
d) coaxially extruding a second thermoplastic having a second melting point T2 around the cable to provide an insulated cable having the extruded second thermoplastic as an outer insulation;
e) cutting the insulated cable to a length that corresponds to the lead length, to provide an insulated lead cable;
f) providing a plurality of electrodes;
g) creating a plurality of contact openings in the vicinity of a distal end of the insulated lead cable, wherein, in order to create a contact opening, parts of the outer insulation and parts of the insulating layer of at least one of the electrically conductive channels are removed so that the at least one electrically conductive channel can be selectively contacted via each of the contact openings;
h) electrically contacting the plurality of electrodes with the plurality of electrically conductive channels via the plurality of contact opening, wherein each of the electrodes is selectively electrically connected to at least one of the electrically conductive channels via one of the contact openings;
wherein the first melting point T1 and the second melting point T2 have a temperature difference DT of greater than or equal to 30°C.

2. The method according to claim 1, wherein the first melting point T1 is higher than the second melting point T2.

3. The method according to claim 1, wherein the first melting point T1 is in a range between 230°C and 290°C.

4. The method according to claim 1, wherein the second melting point T2 is in a range between 150°C and 260°C.

5. The method according to claim 1, wherein the first thermoplastic comprises a fluorinated polymer.

6. The method according to claim 5, wherein the fluorinated polymer is an ethylene-tetrafluoroethylene copolymer (ETFE), a perfluoroalkoxy polymer (PFA), a polyvinylidene fluoride (PVDF) and/or a tetrafluoroethylene-hexafluoropropylene copolymer (FEP).

7. The method according to claim 1, wherein the second thermoplastic is a polyurethane (PU).

8. The method according to claim 7, wherein the polyurethane is a polyester-based or polyether-based polyurethane.

9. The method according to claim 1, wherein the inner lining comprises a polytetrafluoroethylene (PTFE).

10. The method according to claim 1, wherein the extrusion in method step d) is carried out in a temperature range between 170°C and 220°C.

11. The method according to claim 1, wherein the provision in method step b) comprises coaxially extruding the first thermoplastic around the electrical conductor to form the insulating layer.

12. The method according to claim 11, wherein the coaxial extrusion of the first thermoplastic around the electrical conductor is carried out in a temperature range between 250°C and 300°C.
